(19) United States
(12) Plant Patent Application Publication
(10) Pub. No.: US 2005/0217000 P1
(43) Pub. Date: Sep. 29, 2005

(54) PETUNIA PLANT NAMED 'SUNCOPABLUE'

(50) PETUNIA HYBRIDA; PETUNIA

(76) Inventors: Takuro Ishihara, Higashikurume (JP); Kazunari Iwaki, Omihachiman (JP); Kiyoshi Miyazaki, Hikone (JP); Shinya Miyano, Katori-gun (JP)

Correspondence Address: CHRISTIE, PARKER & HALE, LLP, PO BOX 7068, PASADENA, CA, 91109-7068, US

(21) Appl. No.: 10/811,242

(22) Filed: Mar. 25, 2004

(51) Int. Cl. A01H 5/00

(52) U.S. Cl. PLT/356

(57) ABSTRACT

Disclosed herein is a new and distinct variety of Petunia plant having a decumbent habit and short stems. The Petunia plant has abundant branching, and a great profusion of blooms, the whole plant remaining in bloom for a considerable period of time. The flowers are single and small, the petals having vivid purple color. The inside color of the corolla throat is brilliant purple and the outside of the corolla tube is moderate purple. The plant exhibits high resistance to heat, cold, rain and disease.

### BOTANICAL/COMMERCIAL CLASSIFICATION

Petunia hybrida/Petunia Plant

### VARIETAL DENOMINATION

cv. ‘Suncopablue’

### BACKGROUND OF THE VARIETY

The present invention relates to a new and distinct variety of Petunia plant originated from crossing of a seedling called ‘Fantasy Blue’ as the female parent and a Petunia hybrid variety called ‘PAB2’ as the male parent.

The Petunia is a very popular plant that is used for flower bedding and potting in the summer season. There are only a few Petunia varieties which do not have an upright growth habit and which have a high resistance to rain, heat, and disease. Petunias of the ‘Revolution’ series include ‘Revolution Purple pink’ (U.S. Plant Pat. No. 6,915), ‘Revolution Brilliant pink’ (U.S. Plant Pat. No. 6,914), ‘Revolution Brilliantpink-Mini’ (U.S. Plant Pat. No. 6,899), and ‘Revolution Blue vein’ (U.S. Plant Pat. No. 9,322). These are decumbent type plants having long stems, a lower plant height, abundant branching, and a high resistance to heat, rain and disease. However, there are only a few Petunia varieties having a decumbent and compact plant shape, a great profusion of small size flowers, vivid purple petals and a high resistance to rain, heat, and disease. Accordingly, this invention was aimed at obtaining a new Petunia variety having vivid purple petals, together with the above features.

Progress

The female parent ‘Fantasy Blue’ used in the crossing of ‘Suncopablue’ is a cultivar, having an erect growth habit with many branches. It has small single flowers, the petals having vivid violet color. The seed of ‘Fantasy Blue’ was bought from Sakata Seed Corp.

The male parent ‘PAB2’ used in the crossing of ‘Suncopablue’ is a strain of our breeding lines, having a decumbent growth habit with many branches. It has small single flowers, the petals having a violet color.

In July 2000, crossing of ‘Fantasy Blue’ as the female parent and ‘PAB2’ (unpatented) as the male parent was conducted at Yokaichi-shi, Shiga-ken, Japan. In April 2001, 80 seedlings were obtained from that crossing. These seedlings were grown in pots in glasshouses and were evaluated. One seedling was selected in view of its growth habit, flower size and color in September 2001. That seedling was propagated by cutting and a trial was carried out by flower potting and bedding from April to September 2002 at Yokaichi-shi, Shiga-ken, Japan. The botanical characteristics of that plant were then examined, using similar varieties ‘Revolution Violet-mini’ and ‘Fantasy Crystal Red’ for comparison. As a result, it was concluded that this Petunia plant is distinguishable from any other variety, whose existence is known to us, and is uniform and stable in its characteristics. Then the new variety of Petunia plant was named ‘Suncopablue’.

In the following description, the color-coding is in accordance with the Horticultural Colour Chart of The Royal Horticultural Society, London, England (R.H.S. Colour Chart).

### SUMMARY OF THE VARIETY

This new variety is unlike any Petunia commercially available as evidenced by the following unique combinations of characteristics.

1. Rather compact and decumbent growth habit with short stems.

2. Having abundant branching and a great profusion of blooms.

3. The flowers are single and small. The petal color is vivid purple (R.H.S. N87A).

4. The plant has a high resistance to rain, cold, heat and disease.

The new variety ‘Suncopablue’ differs from the similar variety ‘Revolution Violet-mini’ in the following points.

1. The spreading area of ‘Suncopablue’ is smaller than that of ‘Revolution Violet-mini’.

2. The stem length of ‘Suncopablue’ is shorter than that of ‘Revolution Violet-mini’.

3. The internode length of ‘Suncopablue’ is shorter than that of ‘Revolution Violet-mini’.

4. The flower size of ‘Suncopablue’ is smaller than that of ‘Revolution Violet-mini’.

The new variety ‘Suncopablue’ differs from the similar variety ‘Fantasy Crystal Red’ in the following points.

1. The growth habit of ‘Suncopablue’ is decumbent. That of ‘Fantasy Crystal Red’ is erect.

2. The spreading area of ‘Suncopablue’ is larger than that of ‘Fantasy Crystal Red’.

3. The stem length of ‘Suncopablue’ is shorter than that of ‘Fantasy Crystal Red’.

4. The leaf of ‘Suncopablue’ is thinner than that of ‘Fantasy Crystal Red’.

5. The petal color of ‘Suncopablue’ is vivid purple (R.H.S. N87A). That of ‘Fantasy Crystal Red’ is vivid red (R.H.S. 52A) with strong red (R.H.S. 53C) vein.

6. The apex shape of petal of ‘Suncopablue’ is rounded. That of ‘Fantasy Crystal Red’ is obtuse.

7. The petal lobation of ‘Suncopablue’ is shallower than that of ‘Fantasy Crystal Red’.

8. The heat resistance of ‘Suncopablue’ is stronger than that of ‘Fantasy Crystal Red’.

The new variety of Petunia plant ‘Suncopablue’ was asexually reproduced by the use of cuttings at Yokaichi-shi, Shiga-ken, Japan, and homogeneity and stability thereof were confirmed. The instant plant retains its distinctive characteristics and reproduces true to type in successive generations.

### BRIEF DESCRIPTION OF THE PHOTOGRAPH

The depicted plants had been reproduced by the use of cuttings and were photographed during July 2003 while cultivating under the trial field in 15 cm pots at an age of approximately 6 months at Yokaichi-shi, Shiga, Japan.

FIG. 1 illustrates a typical plant of the new variety of Petunia plant ‘Suncopablue’ while growing in a pot.

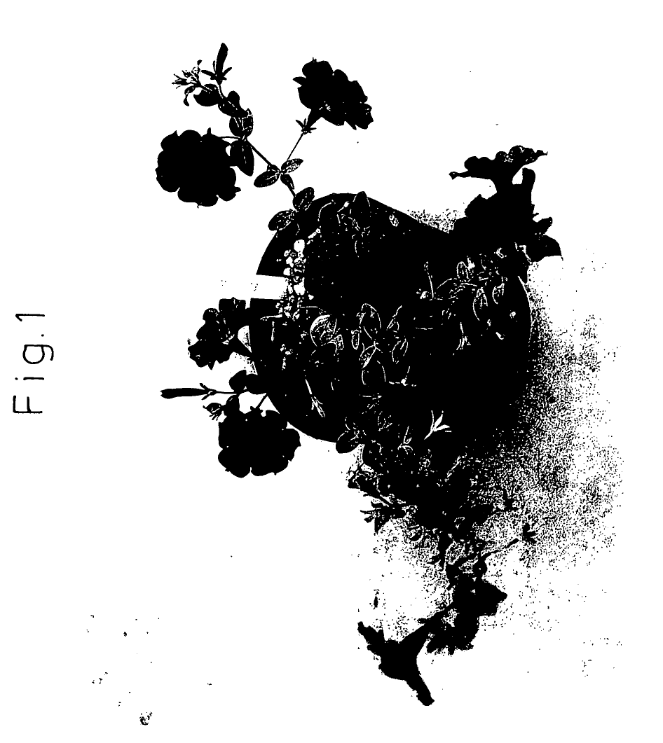

FIG. 2 illustrates a close view of typical foliage and blossoms of the new variety of Petunia plant ‘Suncopablue’.

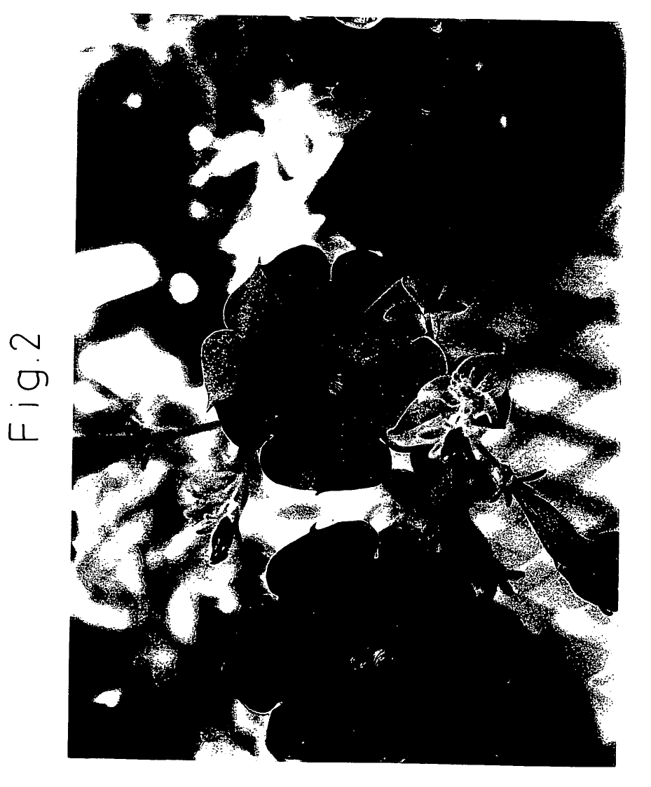

### DESCRIPTION OF THE VARIETY

The botanical characteristics of the new and distinct variety of Petunia plant named ‘Suncopablue’ are as follows when observed during July at Yokaichi-shi, Shiga-ken, Japan, at an age of approximately 6 months.
Plant:
   Growth habit.—Decumbent.
   Plant height.—Approximately 15.6 cm.
   Spreading area of plant.—Approximately 26.0 cm.
   Blooming period.—Early April to late October in the southern Kanto area, Japan. The plant shape does not change throughout this period. A typical flower commonly lasts approximately 5 days on the plant when experiencing a temperature of approximately 20° C.
Stem:
   Length.—Approximately 2.4 cm.
   Thickness.—Approximately 1.5 mm.
   Pubescence.—Normal.
   Branching.—Abundant.
   Internode length.—Approximately 2.1 cm.
   Color.—R.H.S. 144A (strong yellow green).
Leaf:
   Whole shape.—Elliptic with entire margin. The apex shape is acute, and the base shape is attenuate.
   Length.—Approximately 3.2 cm.
   Width.—Approximately 2.2 cm.
   Color.—Upper side color is R.H.S. 144B (vivid yellow green). Lower side color is R.H.S. 146C (moderate yellow green).
   Thickness.—Approximately 0.2 mm.
   Pubescence.—Sparse.
Flower:
   Facing direction.—Slanted upward.
   Type.—Single.
   Shape.—Funnel-shaped, with five-fissures.
   Shape of petal tip.—Rounded.
   Lobation.—Shallow.
   Waving of petal.—Weak.
   Diameter.—Approximately 4.4 cm.
   Color.—Petal; R.H.S. N87A (vivid purple). Inside color of the corolla throat; R.H.S. N81C (brilliant purple). Outside color of the corolla tube; R.H.S. 83B (moderate purple).
   Reproductive organs.—1 normal pistil and 5 normal stamens. Color of pistil is R.H.S. 15C (light yellow green). Color of stamen is R.H.S. 150D (pale yellow green).
   Peduncle.—Approximately 0.8 mm in diameter and Approximately 1.8 cm in length.
   Calyx.—Narrow. 5 sepals in fused at the base.
   Physiological and ecological characteristics.—High resistance to cold, rain, heat and disease. Moderate resistance to pests.

This new variety of Petunia plant is most suitable for flower bedding and potting, particularly in hanging pots or planters. Pinching of old blossoms will enhance the formation of new blossoms.

1. A new and distinct variety of Petunia plant named ‘Suncopablue’, substantially as herein illustrated and described.
